(19) United States
(12) Patent Application Publication
(10) Pub. No.: US 2024/0392322 A1
(43) Pub. Date: Nov. 28, 2024

(54) Nucleotides Comprising CasPhi Modified for Nuclear Targeting

(71) Applicant: Vanderbilt University, Nashville, TN (US)

(72) Inventors: Tonia S. Rex, Nashville, TN (US); Jon R. Backstrom, Nashville, TN (US)

(21) Appl. No.: 18/685,204

(22) PCT Filed: Aug. 25, 2022
(86) PCT No.: PCT/US22/41561

§ 371 Date: Feb. 20, 2024

Related U.S. Application Data
(60) Provisional application No. 63/237,077, filed on Aug. 25, 2021.

(51) Int. Cl. C12N 15/90; C12N 9/22; C12N 15/11

(52) U.S. Cl. CPC C12N 15/907; C12N 9/22; C12N 15/11; C12N 2310/20

(57) ABSTRACT

The presently-disclosed subject matter relates to a compositions and methods that makes use of a CasΦ (CasPhi) that has been modified for effective nuclear targeting. In particular, certain embodiments of the presently-disclosed subject matter relate to unique nucleic acid molecules, compositions, and methods for delivery of CasPhi to the nucleus to effectively modulate expression or otherwise target of a gene of interest. A nucleic acid disclosed herein includes a nucleotide encoding a CasPhi, wherein there are no amino acid substitutions, or wherein proline residues at amino acids 749 and 753 have been substituted; a nucleotide encoding an amino-terminal linker connected to the amino-terminal end of the CasPhi; a nucleotide encoding a carboxy-terminal linker connected to the carboxy-terminal end of the CasPhi; and a nucleotide encoding a nuclear localization signal (NLS) downstream from the CasPhi and carboxy-terminal linker.

### RELATED APPLICATIONS

This application claims priority from U.S. Provisional Application Ser. No. 63/237,077 filed Aug. 25, 2021, the entire disclosure of which is incorporated herein by this reference.

### GOVERNMENT INTEREST

This invention was made with government support under grant number DoD W81XWH-17-2-0055, NEI ROIEY022349, NEI U24EY29893, NEI P30EY008126 awarded by the Department of Defense and the National Institutes of Health. The government has certain rights in the invention.

### TECHNICAL FIELD

The presently-disclosed subject matter generally relates to a compositions and methods that makes use of a CasΦ (CasPhi) that has been modified for effective nuclear targeting. In particular, certain embodiments of the presently-disclosed subject matter relate to unique nucleic acid molecules, compositions, and methods for delivery of CasPhi to the nucleus to effectively modulate expression or otherwise target of a gene of interest.

### INTRODUCTION

Adeno-associated virus (AAV) has a number of advantages in the clinical context, for example, capsid serotype increases targeting specificity, it is unable to replicate, it does not integrate into the genome of post-mitotic cells, and it is FDA-approved and clinically available for the treatment of, for example, Lebers Congenital Amaurosis due to mutations in Rpe65.

Due to the ˜4.7 kb packaging size limitation of AAV, it can be challenging to incorporate certain systems in a complete and functional form. An example of this challenge is in connection with CRISPR constructs. Although a SadCas9 CRISPRi construct has been packed into a single AAV that was functional in vivo (1), a small promoter was required. Meanwhile, the gold standard for delivery of functional CRISPR machinery requires two AAV particles: one with SadCas9-KRAB and the other with a U6 polymerase promoter driving expression of a guide RNA (gRNA) that targets a gene of interest (24).

There are various disadvantages with requiring multiple AAVs. Particularly, AAV is inefficient, particularly for transductions of neurons. The likelihood of transducing sufficient numbers of cells to enact a clinical effect with multiple AAVs is extremely low. Higher titers of AAV would be needed, decreasing the safety of the approach.

As an alternative, there have been some laboratory attempts to perform Cas-mediated gene editing to permanently block transcription of a pathological gene. While this is advantageous in gain of function inherited degenerations, if off target effects occur they are permanent. Thus, this is a risky approach for clinical therapy.

The significant discovery of smaller bacteriophage CasPhi proteins (2) provides an opportunity to include larger cell-type selective promoters and/or more than one guide RNA (gRNA) to target clinically-relevant genes.

However, whether utilizing AAV delivery, or delivery by another vector or particle, as with proteins such as Streptococcus aureus Cas9 (saCas9), the CasPhi protein is lacking in its capacity for nuclear localization.

### SUMMARY

The presently-disclosed subject matter meets some or all of the above-identified needs, as will become evident to those of ordinary skill in the art after a study of information provided in this document.

As disclosed herein, the presently-disclosed subject matter relates to unique nucleic acid molecules and a system for delivery of dCasPhi-based CRISPR to the nucleus to effectively modulate expression or otherwise target of a gene of interest.

The presently-disclosed subject matter includes nucleic acid molecules that comprises (a) a nucleotide encoding a CasPhi, wherein there are no amino acid substitutions, or wherein proline residues at amino acids 749 and 753 have been substituted: (b) a nucleotide encoding an amino-terminal linker connected to the amino-terminal end of the CasPhi: (c) a nucleotide encoding a carboxy-terminal linker connected to the carboxyl-terminal end of the CasPhi; and (d) a nucleotide encoding a nuclear localization signal (NLS) downstream from the CasPhi and carboxy-terminal linker.

In some embodiments, the nucleic acid also includes a repressor domain or an activator domain. In some embodiments, the nucleic acid includes a repressor domain downstream from the CasPhi and a carboxy-terminal linker. In some embodiments, the repressor domain is between the carboxy-terminal linker and the NLS. In some embodiments, NLS is between the carboxy-terminal linker and the repressor domain.

In some embodiments, the nucleic acid also includes a first promoter operably connected to the repressor domain or the dCasPhi. In some embodiments, the nucleic acid also includes a guide RNA (gRNA) and a second promoter operably connected to the gRNA.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned: likewise, those features can be applied to other embodiments of the presently-disclosed subject matter, whether listed in this Summary or not.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1D. Cellular distribution of transiently expressed dCasPhi constructs. Cells were labeled with antibodies against the HA-tag (red) of dCasPhi and DAPI (blue) to detect nuclei. Predominant labeling of dCasPhi was either cytoplasmic (C) without discernible nuclear detection (FIG. 1A); nuclear and cytoplasmic (N/C) (FIG. 1B); punctate cytoplasmic (PC) (FIG. 1C); or nuclear (N) (FIG. 1D). Representative constructs are shown below each panel. The results are from at least three independent experiments with >90% of the HA-positive cells displaying the indicated labeling pattern.

FIGS. 2A-2I. Analysis of dCasPhi-2 CRISPRi cell lines.

FIG. 2A. Schematic of the four dCasPhi CRISPRi constructs used in the study.

FIG. 2B. Location and orientation of the four gRNAs (VJ-1 to-4, yellow arrows) that target mVEGF and the hypoxia-response element (HRE; grey box). Reference sequence NC 000083.7 was used to determine the sequence and position of gRNAs and reference sequence NM_001287056 was used to identify the transcription start site (curved, black arrow).

FIG. 2C. Predominant nuclear localization of Dox-induced dCasPhi (construct 829), scale bar indicates 100 μM.

FIGS. 2D and 2E. Comparison of parental MMT cells and polyclonal cell lines expressing construct 829 with the control gStop or four mVEGF gRNAs. FIG. 2D includes the effect of inducing dCasPhi protein expression (Dox/SF) on levels of basal, secreted VEGF. FIG. 2E includes the effect of FG-4592-mediated hypoxia (Dox+FG/Dox) on levels of hypoxic, secreted VEGF.

FIG. 2F. Comparison between 827/VJ-1 and 829/VJ-1 CasPhi constructs on hypoxic induction of secreted VEGF.

FIG. 2G. Effect of hypoxia on levels of VEGF and AngPTL4 mRNA from parental cells or 829 cell lines expressing gStop or VJ-1.

FIGS. 2H and 2I. Comparison of dCasPhi-ZIM3 mRNA (FIG. 2H) and protein (FIG. 2I) from cell lines expressing 829/gStop or 829/VJ-1.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is based, at least in part, upon the following discoveries: an altered CasΦ (CasPhi) and CasPhi-containing construct to result in nuclear localization of the protein: a design to fuse a repressor domain (e.g., an inhibitory Krüppel-associated box domain (KRAB-domain: e.g., ZIM3)) onto a deactivated CasPhi (dCasPhi) to maintain nuclear localization and accentuate knock-down effect: a guide RNA sequence that works with CasPhi construct to knock-down basal levels of VEGF (exemplary target) mRNA and protein; and a guide RNA sequence that works with CasPhi construct to knock-down induced, but not basal, levels of VEGF (exemplary target) mRNA and protein.

The presently-disclosed subject matter includes nucleic acid molecules as described herein, as well as polypeptide molecules that are encoded by any of the nucleic acid molecules as disclosed herein.

The presently-disclosed subject matter includes a nucleic acid that comprises (a) a nucleotide encoding a CasPhi, wherein there are no amino acid substitutions, or wherein proline residues at amino acids 749 and 753 have been substituted: (b) a nucleotide encoding an amino-terminal linker connected to the amino-terminal end of the CasPhi: (c) a nucleotide encoding a carboxy-terminal linker connected to the carboxyl-terminal end of the CasPhi; and (d) a nucleotide encoding a nuclear localization signal (NLS) downstream from the CasPhi and carboxy-terminal linker.

As used herein, CasPhi wherein there are no amino acid substitutions refers to the amino acid sequence as disclosed by Doudna, et al. (2020) Science (2), and additional details regarding the sequence can be found at www.addgene.org/Jennifer_Doudna/. In embodiments where the CasPhi includes amino acid substitutions, the residue number of the amino acid is made with reference to the amino acid sequence as disclosed by Doudna, et al. (2020) Science (2). In embodiments where the CasPhi includes amino acid substitutions, proline residues at amino acids 749 and 753 are substituted for amino acids that will allow for the bend associated with proline to be removed. For example, alanine or glycine could be used. In some embodiments, in the CasPhi the proline residues at amino acids 749 and 753 have been substituted with alanine (P749A and P753A). In some embodiments, the CasPhi is catalytically inactivated.

In some embodiments, the nucleic acid also includes a nucleotide encoding a protein domain for facilitating a CRISPR application. As will be recognized by the skilled artisan, such applications can include, for example, gene editing, imaging, transcriptional activation, and transcriptional repression.

In some embodiments, the nucleic acid also includes a repressor domain or an activator domain. CRISPR interference (CRISPRi) makes use of a CasPhi bound to repressor that, together with a guide RNA, repress or decrease transcription of a target gene. In some embodiments, the nucleic acid includes a repressor domain downstream from the CasPhi and a carboxy-terminal linker. In some embodiments, the repressor domain is between the carboxy-terminal linker and the NLS. In some embodiments, NLS is between the carboxy-terminal linker and the repressor domain. In some embodiments, the repressor domain is selected from the group consisting of KRAB, SRDX, T1R1, MAD1, and TIEG1. In some embodiments, the repressor domain is a KRAB-domain fusion. In some embodiments, the KRAB-domain fusion is selected from the group consisting of ZNF10 or ZIM3. In some embodiments, the nucleic acid also includes a second repressor domain. In some embodiments, the second repressor domain is connected to the amino-terminal end of an amino-terminal linker.

As noted above, embodiments of the nucleic acid include an amino-terminal linker and/or a carboxy-terminal linker. In some embodiments, the amino-terminal linker consists of about 5, 6, 7, 8, 9, 10, 11, 12, 13, 14, or 15 nucleotides. In some embodiments, the amino-terminal linker comprises the sequence GGSGGGS (SEQ ID NO: 1). In some embodiments, the carboxy-terminal linker consists of about 5, 6, 7, 8, 9, 10, 11, 12, 13, 14, or 15 nucleotides. In some embodiments, the carboxy-terminal linker comprises the sequence (GGGGS (SEQ ID NO: 2))x, wherein x is 2 or 3.

As also noted above, embodiments of the nucleic acid include an NLS. In some embodiments, the NLS comprises the sequence PAAKRVKLD (SEQ ID NO: 3) (myc-NLS). In some embodiments, the NLS comprises the sequence PKKKRKV (SEQ ID NO: 4) (SV40-NLS).

In some embodiments, the nucleic acid also includes a first promoter operably connected to the repressor domain or the dCasPhi. In some embodiments of the nucleic acid molecule, the first promoter is operably connected to the nucleotide encoding the repressor domain. In some embodiments, the first promoter an RNA polymerase II promoter. In some embodiments, the first promoter an RNA polymerase II promoter of about 25 to about 400 nucleotides in length. In some embodiments, the first promoter is about 350 to about 375 nucleotides in length. In some embodiments, the first promoter optionally includes an enhancer. As is well-known in the art, a promoter is a nucleotide sequence where transcription of an operatively-connected gene is initiated. Promoters can include an RNA polymerase binding site and transcription factor binding sites. In some cases, an enhancer is additionally provided. As is well-known in the art, an enhancer is a nucleotide sequence that can be bound by transcription factors and activators to enhance likelihood of transcription of an operatively-connected gene.

In some embodiments, the nucleic acid also includes a guide RNA (gRNA) and a second promoter operably connected to the gRNA. In some embodiments, the second promoter is an RNA polymerase II promoter. In some embodiments of the nucleic acid molecule, the second promoter is an RNA polymerase II promoter of about 150-250 nucleotides (base pairs) in length.

As will be recognized by the skilled artisan, guide-RNA (gRNA) is a guide-polynucleotide including ribonucleotides and at least a guide-sequence that is able to hybridize with a target-polynucleotide and is able to direct sequence-specific binding of the RNA-guided nuclease system to a target-polynucleotide. In this regard, gRNA can be described as a fusion of sequences, including a sequence for CasPhi binding, which can be referred to as a gRNA scaffold sequence, and a sequence for directing a Cas-gRNA complex to a target DNA, which can be referred to as a gRNA targeting sequence.

Some embodiments of the nucleic acid molecule further include a poly A domain. In some embodiments, the poly A domain is an SV40 polyA domain. In some embodiments, the poly A domain is about 115 to about 130 nucleotides (base pairs) in length.

In some embodiments, a nucleic acid molecule or polypeptide molecule of the presently-disclosed subject matter is represented as set forth in Table 1 hereinbelow.

The presently-disclosed subject matter further includes a composition comprising a nucleic acid or polypeptide as disclosed herein, and a component for delivery. In some embodiments, the delivery is particle or nanoparticle delivery, PEG-mediated delivery, bombardment mediated delivery, or agrobacterium-mediated delivery.

The presently-disclosed subject matter further includes a vector comprising the nucleic acid as disclosed herein. In some embodiments, the vector is selected from an adeno-associated virus (AAV) vector, adenoviral (AdV) vector, a lentivirus (LV) vector, and a bacteriophage.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Study Overview and Example of Target Used to Establish Utility of Construct

The size of bacterial Cas proteins is a limiting factor for epigenetic gene interference (CRISPRi) constructs when packaged in adeno-associated viruses (AAVs), an FDA-approved vector for translational studies. Although bacteriophage Cas proteins (CasPhi) are smaller than bacterial Cas proteins, prior to the information disclosed herein, it was unknown if deactivated/dead CasPhi (dCasPhi) could accommodate fused transcriptional domains, traffic to the nucleus, and modify gene expression.

As disclosed herein, it was found that dCasPhi has a limited capacity for amino-terminal peptides but allowed the inclusion of larger carboxyl-terminal CRISPRi KRAB-domain fusions such as ZNF10 or ZIM3.

Tet-ON transposons with dCasPhi-ZIM3 were used to evaluate the efficacy of VEGF gRNAs against basal and hypoxia-induced (FG-4592) levels of VEGF from mouse mammary tumor cell lines. As disclosed herein, a gRNA was discovered, which knocks-down only hypoxia-induced VEGF expression.

The results presented herein establish that dCasPhi (P749A/P753A)-ZIM3/U6: gRNA CRISPRi components with a size of 3.3 kb are functional for gene interference, providing a framework to include large promoters and/or multiple gRNAs within the ˜4.5 kb size limitation of AAV.

### Example 2: Trafficking of dCasPhi to the Nucleus

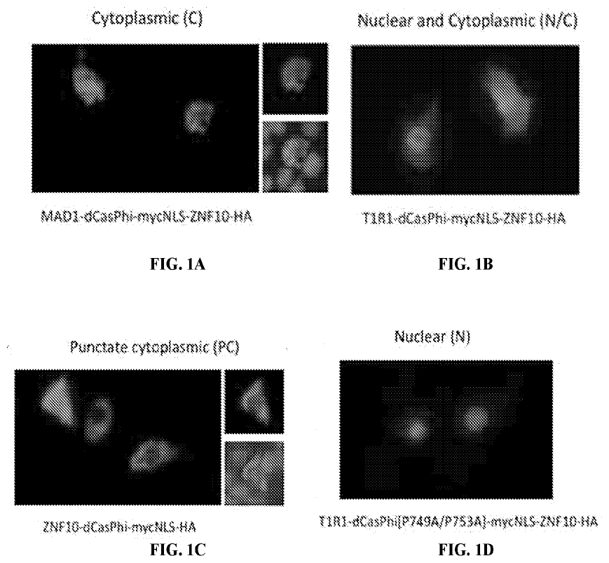

The first set of studies evaluated the effectiveness of rigid or flexible linkers between the carboxyl-terminus of HA-tagged dCasPhi-2 (HA-dCasPhi) and the C′-Myc nuclear localization signal (mycNLS, Table 1, Section A). Cellular localization was scored as either cytoplasmic (C), meaning that nuclear labeling was not detected above cytoplasmic levels, nuclear and cytoplasmic (N/C), punctate cytoplasmic (PC), or nuclear (N). Representative images of these labeling patterns using anti-HA antibodies are presented in FIG. 1. The rigid, helical linker termed EA with 3 (EA-S), 4 (EA-M), or 5 (EA-L) helices (5) was unable to provide nuclear localization of HA-dCasPhi. Cytoplasmic localization of HA-dCasPhi was also observed with the 5-amino acid flexible, glycine-rich linker (GS-S). However, a 10-amino acid flexible linker (GS-M) provided a mixed nuclear/cytoplasmic (N/C) labeling pattern. HA-dCasPhi with the 15-amino acid flexible linker (GS-L) showed an identical nuclear/cytoplasmic pattern, and to be conservative, this linker was used at the carboxyl-terminus of all subsequent dCasPhi constructs.

The effects of adding amino-terminal domains were evaluated next. A small set of experiments were unable to document a functional effect of amino-terminal mycNLS (Table 1, Section B); therefore, all subsequent constructs utilized the NLS at the carboxyl-terminus of dCasPhi. Since nuclear/cytoplasmic localization of dCasPhi was observed with the HA-tag placed at either the amino- or carboxyl-terminus, the effects of adding transcriptional repressor domains to dCasPhi-mycNLS-HA was examined (Table 1, Section C). These included peptides from either the R1 domain of TIEG1 (T1R1: (6)) or MAD1 (7,8), or the much larger KRAB protein domain of ZNF10 (911). Only the T1R1 construct (FIG. 1B) displayed a similar nuclear/cytoplasmic localization pattern as HA-dCasPhi. Substitution of T1R1 with the MAD1 peptide resulted in less nuclear labeling (FIG. 1A) whereas inclusion of the ZNF10 KRAB domain resulted in a punctate, cytoplasmic distribution (PC, FIG. 1C).

The effect of amino-terminal T1R1 or MAD1 peptides was examined in constructs with carboxyl-terminal ZNF10 (Table 1, Section D). The labeling patterns of constructs with T1R1 (nuclear/cytoplasmic) or MAD1 (cytoplasmic) were identical to those without ZNF10, irrespective of the placement of ZNF10 at the carboxyl-terminus of dCasPhi (i.e. dCasPhi-ZNF10-mycNLS-HA or dCasPhi-mycNLS-ZNF10-HA). Because T1R1 constructs were permissive to at least partial steady-state nuclear localization, subsequent trafficking studies utilized the T1R1 domain at the amino-terminus of dCasPhi.

Examination of the carboxyl-terminal region of CasPhi-2 (2) revealed the presence of 4 proline residues within the last 18 amino acids that were not conserved in other CasPhi proteins. Combinations of these proline residues were mutated to assess their effects on dCasPhi trafficking (Table 1, Section E). The T1R1-dCasPhi-mycNLS-ZNF10-HA construct was used for these studies because the order of NLS and ZNF10 did not influence trafficking (Table 1, Section D), and the transcriptional repressor domain may be more accessible for its function in this orientation. Whereas a single P753A mutation had no effect on altering the nuclear/cytoplasmic distribution, the combined P749A/P753A mutations resulted in predominant nuclear labeling of dCasPhi (FIG. 1D). Neither P740G/P741G nor P740G/P741G/P749A/P753A mutations enhanced nuclear localization of dCasPhi, documenting a clear preference of P749A/P753A mutations for providing accessibility of ZNF10-based CRISPRi construct to the nuclear import machinery, based on its predominant nuclear localization versus the other tested constructs.

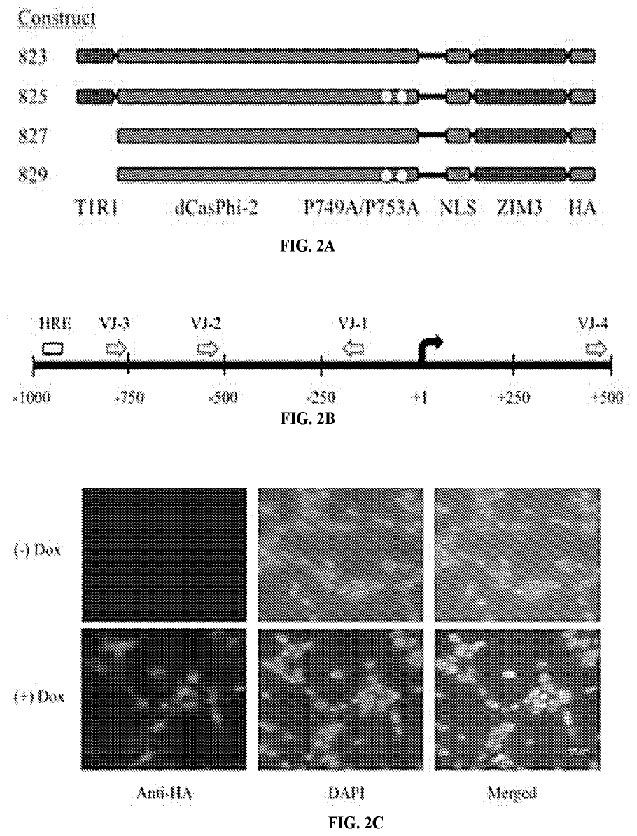

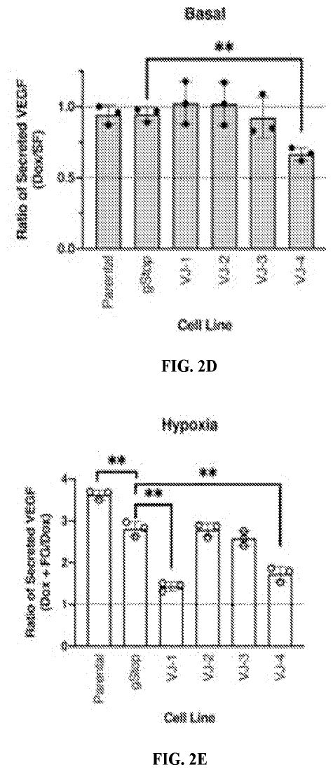

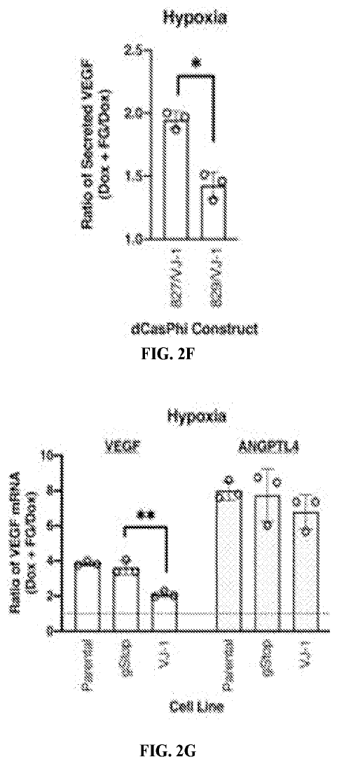

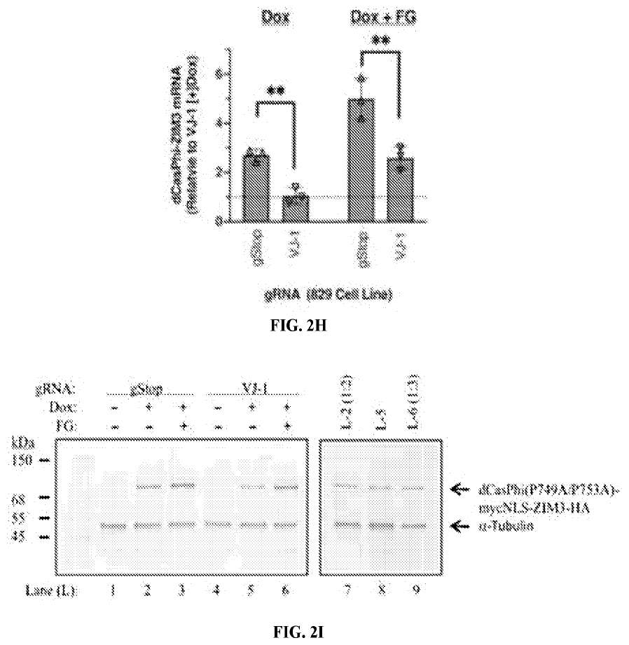

Alerasool et al. (12) evaluated transcriptional repression with several KRAB domains and found that ZIM3 displayed significantly greater efficacy than ZNF10. Therefore trafficking of CasPhi-ZIM3 constructs was also examined (Table 1, Section F). Both wild-type dCasPhi within T1R1-dCasPhi-mycNLS-ZIM3-HA (referred to as 823; FIG. 2A) and dCasPhi with P749A/P753A mutations (T1R1-dCasPhi (P749A/P753A)-mycNLS-ZIM3-HA, referred to as 825) showed predominant nuclear labeling patterns.

With regard to the selection of NLS, studies were conducted using SV40 in which cytoplasmic labeling resulted (data not shown). Accordingly. NLS that are not SV40, such as myc, contemplated for use.

TABLE 1
Summary of dCasPhi-2 constructs and their corresponding
cellular localization. The features in bold are
compared between respective constructs.

| | Local- |
|---|---|
| | ization² |
| A) Carboxyl-Terminal Linker¹ | |
| HA-(mGS)-dCasPhi-(EA-S)-mycNLS | C |
| HA-(mGS)-dCasPhi-(EA-M)-mycNLS | C |
| HA-(mGS)-dCasPhi-(EA-L)-mycNLS | C |
| HA-(mGS)-dCasPhi-(GS-S)-mycNLS | C |
| HA-(mGS)-dCasPhi-(GS-M)-mycNLS | N/C |
| HA-(mGS)-dCasPhi-(GS-L)-mycNLS | N/C |
| B) Amino-Terminal mycNLS³ | |
| mycNLS-(mGS)-dCasPhi-HA | C |
| mycNLS-(EA-L)-T1R1-(mGS)-dCasPhi-HA | C |
| mycNLS-(EA-L)-MAD1-(mGS)-dCasPhi-HA | C |
| C) Amino-Terminal Constructs, Carboxyl-Terminal mycNLS³ | |
| dCasPhi-mycNLS-HA | N/C |
| T1R1-(mGS)-dCasPhi-mycNLS-HA | N/C |
| MAD1-(mGS)-dCasPhi-mycNLS-HA | C |
| ZNF10-(mGS)-dCasPhi-mycNLS-HA | PC |
| D) Carboxyl-Terminal ZNF10^{3, 4} | |
| MAD1-dCasPhi-ZNF10-mycNLS-HA | C |
| MAD1-dCasPhi-mycNLS-ZNF10-HA | C |
| T1R1-dCasPhi-ZNF10-mycNLS-HA | N/C |
| T1R1-dCasPhi-mycNLS-ZNF10-HA | N/C |
| E) dCasPhi Carboxyl-Terminal Proline Mutations^{3, 4} | |
| T1R1-dCasPhi-mycNLS-ZNF10-HA | N/C |
| T1R1-dCasPhi(P753A)-mycNLS-ZNF10-HA | N/C |
| T1R1-dCasPhi(P749A/P753A)-mycNLS-ZNF10-HA | N |
| T1R1-dCasPhi(P740G/P741G)-mycNLS-ZNF10-HA | N/C |
| T1R1-dCasPhi(P740G/P741G/P753/P749A)-mycNLS- | N/C |
| ZNF10-HA | |
| F) Carboxyl-Terminal ZIM3^{3, 4} | |
| T1R1-dCasPhi-mycNLS-ZIM3-HA | N |
| T1R1-dCasPhi(P749/753A)-mycNLS-ZIM3-HA | N |

¹Linkers between the carboxyl-terminus of dCasPhi and the C-Myc nuclear localization signal (mycNLS) included a rigid, helical linker or the flexible linker GS [GGGGSx] (SEQ ID NO: 2). For x, EA-S = 3, EA-M = 4, and EA-L = 5; GS-S = 1, GS-M = 2, and GS-L = 3. The sequence of the modified GS linker (mGS) = [GGSGGGS] (SEQ ID NO: 1).
²Cellular localization of dCasPhi constructs in transiently-transfected mouse AML-12 liver cells. Greater than 90% of cells (n = 3 independent experiments) displayed immunoreactivity predominantly in cytoplasmic (C), a mix of nuclear and cytoplasmic (N/C), punctate cytoplasmic (PC), or nucleus (N). See FIG. 1 for examples of representative cellular localizations.
³All constructs contained the carboxyl-terminal GS-L linker [dCasPhi-(GS-L)]
⁴All constructs contained the amino-terminal mGS linker [mGS-dCasPhi]

### Example 3: Evaluation of gRNAs Against Exemplary Target, VEGF

Four CasPhi-2 gRNAs against mouse VEGF (FIG. 2B) were tested in cell lines derived from mouse mammary tumor (MMT) cells. The gRNAs included regions upstream (VJ-1, VJ-2, and VJ-3) and downstream (VJ-4) of the transcription start site. The negative control gRNA gStop terminates transcription after the CasPhi RNA loop. The CRISPRi function of the more minimalist, trafficking-amenable construct dCasPhi (P749A/P753A)-mycNLS-ZIM3-HA was addressed first; hereto referred to as 829 (FIG. 2A).

Polyclonal cell lines were made with a Sleeping Beauty transposon that includes a doxycycline (Dox)-inducible Tet-ON promoter to drive expression of dCasPhi-ZIM3 and the constitutive U6 promoter driving expression of CasPhi gRNA. Labeling of cells with anti-HA documented Dox-induced expression of dCasPhi-ZIM3 that was primarily localized in the nucleus (FIG. 2C).

The effects of gRNAs against basal secretion of VEGF were tested by treating cells in serum-free media for 24 h in the absence or presence of Dox and determining the levels of VEGF from the supernatant in an ELISA. The values of VEGF from Dox-treated cells were divided by those from untreated cells (Dox/SF) to determine the effect of upregulating dCasPhi-ZIM3 on basal levels of VEGF (FIG. 2D). Dox did not significantly alter the levels of secreted VEGF from parental MMT cells or cell lines expressing gStop, VJ-1, VJ-2, or VJ-3. In contrast, cells expressing VJ-4 showed a 33±5% decrease in secreted VEGF (p=0.003 vs. gStop). Thus, only VJ-4 decreased basal secretion of VEGF.

Next, cells in serum-free media were treated with either Dox or Dox plus the hypoxia-mimetic FG-4592 (FG, (13)) for 24 h to determine the effect of dCasPhi-ZIM3 on hypoxic induction of VEGF (Dox+FG/Dox, FIG. 2E). Parental MMT cells displayed a 3.6-fold increase in VEGF levels that was higher than the 2.8-fold increase from cells expressing dCasPhi-ZIM3 with gStop (p=0.001). No significant differences in secreted VEGF were detected from cells expressing VJ-2 or VJ-3 relative to gStop. However, significantly lower ratios were observed from cells expressing either VJ-1 (1.4-fold, p<0.001) or VJ-4 (1.7-fold, p<0.001).

### Example 4: Comparison of dCasPhi-ZIM3 Constructs

The identification of a dCasPhi gRNA (VJ-1) that decreases hypoxic, but not basal, levels of VEGF in mouse mammary tumor cells prompted a comparison of the four candidate dCasPhi-ZIM3 constructs (FIG. 2A) with either control gStop or VEGF VJ-1 gRNAs. The two negative control cell lines with T1R1 (823/gStop and 825/gStop) displayed minimal 1.5-fold hypoxic-induction of secreted VEGF, precluding them from further analysis. In contrast, the 827 cell lines showed a similar basal response as 829, but the 827/VJ-1 construct was not as effective as 829/VJ-1 at decreasing hypoxia-mediated secretion of VEGF (FIG. 2F, p=0.03).

### Example 5: Analysis of VEGF mRNA Expression

Levels of mRNA were evaluated from parental cells or cells expressing either 829/gStop or 829/VJ-1 to determine if decreased VEGF secretion was due to CRISPRi activity. Treatment of cells with Dox did not alter basal levels of VEGF mRNA relative to serum-free conditions (Dox/SF) with either gStop (1.07±0.04) or VJ-1 (1.04±0.04) gRNAs, which was indistinguishable from parental MMT cells (1.07±0.07). However, treatment of cells with Dox and FG-4592 (Dox+FG/Dox) resulted in 4-fold (parental=3.8±0.1, 829/gStop=3.6±0.4) or 2-fold (829/VJ-1=2.1±0.2) increases of VEGF mRNA (FIG. 2G), a significant difference between control gStop and VEGF VJ-1 gRNAs (p=0.006). In contrast, parental MMT cells and both 829 cell lines showed similar Dox plus FG-4592-mediated responses of ANGPTL4 (FIG. 2G), another hypoxia-induced gene (14).

### Example 6: Examination of dCasPhi-ZIM3 mRNA and Protein

The levels of dCasPhi-ZIM3 mRNA and protein were evaluated to determine whether the apparent CRISPRi activity 829/VJ-1 was simply due to increased dCasPhi expression relative to 829/gStop. Treatment with Dox resulted in 2.7±0.3-fold higher levels of dCasPhi-ZIM3 mRNA from cells expressing 829/gStop than from cells expressing 829/gStop (FIG. 2H) Unexpectedly, treatment with Dox plus FG-4592 resulted in an additional 2 to 2.5-fold increase of dCasPhi-ZIM3 mRNA from both cell lines. Similar increases of dCasPhi mRNA were also observed from cells only treated with FG-4592 (data not illustrated). Blots probed with anti-HA to detect dCasPhi-ZIM3 and control α-tubulin confirmed the differences in CasPhi protein expression between the cell lines as well as increased levels due to the combined treatment with Dox and FG-4592 (FIG. 2I, lanes 1-6).

Further examination of diluted proteins revealed these differences at the protein level. Identical levels of immunoreactive dCasPhi were observed when soluble proteins from 829/gStop cells treated with Dox were diluted 1:2 (5 μg, FIG. 2H lane 7), 829/VJ-1 cells treated with Dox were undiluted (10 μg, lane 8), and 829/VJ-1 cells treated with Dox and FG-4592 were diluted 1:3 (3.3 μg, lane 9). These results confirmed both the increased levels of Dox-induced dCasPhi mRNA and protein from the negative control gStop cell line relative to the VJ-1 cell line and the additional increased protein levels after treatment with Dox plus FG-4592. Together, these results establish that a dCasPhi-ZIM3 CRISPRi construct with gRNA VJ-1 functionally attenuated hypoxic induction of VEGF mRNA and protein.

### Example 7: Discussion of Studies Using Example Target

Trafficking of dCasPhi-2 to the nucleus was greatly enhanced with a flexible, glycine-rich (GS) linker between the carboxyl-terminus and the mycNLS. A 10-amino acid GS linker was sufficient as a 15-amino-acid GS linker displayed similar nuclear labeling of dCasPhi. In contrast, the rigid, helical linkers used in these studies were unable to expose the MycNLS to the nuclear import machinery, based on predominant cytoplasmic labeling. It is contemplated that this difference is due to linker flexibility rather than only length because flexible GS linkers between proteins are more compacted relative to extended, EA helical linkers and increasing the size of GS linkers did not significantly increase the distance between protein domains (15, 16). These findings with dCasPhi are in stark contrast to the previous results with SadCas9 that was more effectively transported to the nucleus with a helical linker (1). Doudna's group that discovered and characterized CasPhi (2) used a Gly-Ser-Gly linker at the carboxyl-terminus of CasPhi-2 that precedes two copies of the SV40 NLS. Based on the results, it is contemplated that only the second SV40 NLS of their construct is functional. It was also found that dCasPhi-mycNLS alone or with the carboxyl-terminal ZNF10 domain displayed a mixed nuclear/cytoplasmic distribution but inclusion of P749A/P753A mutations resulted in predominant nuclear localization. In contrast, dCasPhi-ZIM3 with or without the proline mutations was trafficked to the nucleus.

The first goal was to determine if dCasPhi could accommodate protein fusions at both the amino- and carboxyl-termini and be efficiently trafficked to the nucleus. The results imply that the capacity to add elements to the amino-terminus is greatly limited. For example, the transcriptional-inhibitory peptide domains of TIEG1 and MAD1 have similar sizes and structures (6-8, 17), yet only constructs with the R1 domain of TIEG1 (T1R1) allowed efficient nuclear localization of T1R1-dCasPhi. The HA-tag at the amino-terminus was detected with antibodies, implying that transcription factor-T1R1 interactions could occur, but this is purely speculative because the mycNLS at the amino-terminus was not functional. Evidence that supports a function of the T1R1 domain of the dCasPhi CRISPRi constructs is not provided, but this warrants further investigation. In this regard, mutations of proline residues at the amino-terminus of CasPhi-2 (Pro-Lys-Pro) may be beneficial for adding peptides/proteins, similar to what was found with proline mutations at the carboxyl-terminus.

Although dCasPhi-ZIM3 constructs were efficiently transported to the nucleus, it was unclear if they provided CRISPRi function. Three mouse VEGF gRNAs were tested upstream of the transcription start site (VJ1-VJ3) and one downstream (VJ-4). A dCas9 VEGF gRNA downstream of the transcription start site was recently found that decreases basal expression of VEGF¹. The rationale for testing VJ-4 gRNA, near the functional dCas) gRNA, was to utilize a dCasPhi gRNA with a reasonable likelihood of success in order to evaluate the function of the CasPhi protein constructs because it would be difficult to discern between non-functional protein and ineffective gRNA. Indeed, VJ-4 effectively decreased basal VEGF secretion similar to what was observed with dCas9.

Current treatments for macular degeneration include anti-VEGF therapy that has the unwanted consequence of decreasing basal VEGF, which is required for cell survival (3), and chronic treatment results in unfavorable outcomes such as complement-mediated damage (18). Thus, the goal was to selectively decrease hypoxic-induction of VEGF. gRNAs were evaluated in mouse MMT cells that secrete high levels of VEGF (19). The rationale was to test gRNAs in cells that secrete high basal levels of VEGF, and not interfere with its expression. It was also assumed that hypoxic induction of VEGF would be similar between different cell types so the results could be translated to clinical manifestations. Interestingly, deletion of a 126 bp region of VEGF DNA that includes the VJ-1 gRNA target site did not interfere with hypoxic induction of VEGF (20), implying that CRISPRi activity with VJ-1 was not due to interfering with a positively-acting hypoxic factor in this region.

The significant discovery of small bacteriophage CasPhi proteins (2) has opened the door to a wide-range of epigenetic opportunities, particularly with delivery of components via AAVs that have a limited packaging capacity. The results provide evidence that transcriptional-modifying domains can be added to the carboxyl-terminus of dCasPhi-2. It is contemplated that dCasPhi-2 with carboxyl-terminal proline mutations (P749A/P753A) has utility for relevant applications.

### Example 8: Cloning of CRISPRi Constructs

The protein sequence of dCasPhi-2 (Doudna) was used to create a codon-optimized gene that was purchased from IDT (Integrated DNA Technologies, Coralville, IA). Other components were generated from PCR with Q5 polymerase (New England Biolabs, Ipswich, MA) or synthetic DNA (IDT or Genewiz: South Plainfield, NJ). DNA oligos for gRNAs included 6-′T's for transcription termination (21). Single-stranded gRNA oligos were treated with T4 polynucleotide kinase as per the manufacturer's recommendations (NEB), annealed, and ligated into Zra1/Xho1 insertion sites. Sequencing of plasmids was performed at Genewiz. All relevant dCasPhi-2 CRISPRi plasmids will be available at Addgene.

### Example 9: Analysis of dCasPhi Trafficking

dCasPhi plasmids in AAV backbones were used to transiently transfect mouse AML cells as described (1). dCasPhi was detected with rabbit antibodies against the HA-tag (CST: Cell Signaling Technologies, Danvers, MA) and nuclei detected with DAPI. Each construct was analyzed at least three times and the localization of each scored according to the results presented in FIG. 1.

### Example 10: Generation of Sleeping Beauty Cell Lines

The mouse mammary tumor cell line MMT was purchased from ATCC (Manassas, VA). Cell lines were generated essentially as described (1) except that Tet-ON plasmids (21) contained the hygromycin resistance gene that was mutated to remove gRNA cloning sites (Zra1/Xho). Cells were selected with 0.5 mg hygromycin/ml of media, which resulted in death of parental cells in 11-14 days.

### Example 11: Analysis of Secreted VEGF

Cells in 12-well plates were washed and incubated in serum-free (SF) media for 24 h. Supernatant was removed and replaced with either SF media, SF media with 1 μg/ml doxycycline (Dox), or SF medium with 1 μg/ml doxycycline and 100 UM FG-4592 (Roxadustat, MedChemExpress, Monmouth Junction NJ: Dox+FG). After 24 h, supernatant was collected and centrifuged at 20,000×g for 15 min at 4° C. Samples were diluted 1:20 in PBS and subjected to a mouse VEGF ELISA (R&D Systems, Minneapolis, MN).

### Example 12: Analysis of mRNA Levels with qPCR

RNA was collected from MMT cells and subjected to qPCR as described (1) using PerfeCTa polymerase (Quantabio, Beverly, MA). mVEGF (23) forward and reverse primers, and dCasPhi-2 forward and reverse primers were used. Three sets of dCasPhi-2 primers were evaluated and only the selected pair resulted in undetectable levels of amplified product from parental MMT cells.

### Example 13: Blots of dCasPhi CRISPRi Constructs

Soluble proteins from cells were used to examine immunoreactive protein as described (1). Protein concentrations were determined with the Pierce BCA assay (ThermoFisher Scientific) using BSA as the protein standard. Blots were probed with rabbit anti-HA to detect dCasPhi and mouse anti α-tubulin (CST) as the loading control, and protein detected with secondary alkaline phosphatase-conjugated antibodies (Jackson ImmunoResearch Laboratories, Inc., West Grove, PA) with NBT and BCIP.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1 Backstrom, J. R., Sheng, J., Wang, M. C., Bernardo-Colón, A., and Rex, T. S. (2020) Optimization of S. Aureus dCas9 and CRISPRi elements for a single adeno-associated virus that targets an endogenous gene. Mol. Ther. Methods Clin. Dev. 19, 139-148
2 Pausch, P., Al-Shayeb, B., Bisom-Rapp, E., Tsuchida, C. A., Li, Z., Cress, B. F., Knott, G. J., Jacobsen, S. E., Banfield, J. F., and Doudna, J. A. (2020) CRISPR-CasΦ from huge phages is a hypercompact genome editor. Science 369, 333-337
3 Kurihara, T., Westenskow, P. D., Bravo, S., Aguilar, E., and Friedlander, M. (2012) Targeted deletion of Vegfa in adult mice induces vision loss. J. Clin. Invest. 122, 4213-4217
4 Mancusco, M. R., Davis, R. Norberg, S. M., O'Brien, S., Sennino, B., Nakahara, T., Yao, V. J., Inai, T., Brooks, P., Freimark, B., Shalinsky, D. R., Hu-Lowe, D. D., and McDonald, D. M. (2006) Rapid vascular regrowth in tumors after reversal of VEGF inhibition. J. Clin. Invest. 116, 261026021
5 Marqusee, S. and Baldwin, R. L. (1987) Helix stabilization by Glu⁻ . . . Lys⁺ salt bridges in short peptides of de novo design. Proc. Natl. Acad. Sci. USA 84, 8898-88902
6 Cook, T., Gebelein, B., Belal, M., Mesa, K., and Urrutia, R. (1999) Three conserved transcriptional repressor domains are a defining feature of Sp1-like zinc finger proteins. J. Biol. Chem. 274, 29500-29504
7 Ayer, D. E., Lawrence, Q. A., Eisenman, R. N. (1995) Mad-Max transcriptional repression is mediated by ternary complex formation with mammalian homologs of yeast repressor Sin3. Cell 80, 767-776
8 Cowley, S. M., Kang, R. S., Frangioni, J. V., Yada, J. J., DeGrand, A. M., Radhakrishnan, I., and Eisenman, R. N. (2004) Functional analysis of the Mad1-mSin3A repressor-corepressor interaction reveals determinants of specificity, affinity, and transcriptional response Mol. Cell. Biol. 24, 2698-2709
9 Bellefroid, E. J., Poncelet, D. A., Lecocq, P. J., Revelant, O., and Martial, J. A. (1991) The evolutionarily conserved Krüppel-associated box domain defines a subfamily of eukaryotic multifingered proteins. Proc. Natl. Acad. Sci. USA 88, 3608-3612
10 Margolin, J. F., Friedman, J. R., Meyer, W. K. H., Vissing, H., Thiesen, H. J., and Rauscher III, F. J. (1994) Krüppel-associated boxes are potent transcriptional repression domains. Proc. Natl. Acad. Sci. USA 91, 4509-4513
11 Witzgall, R., O'Leary, E., Leaf, A., Önaldi, D., and Bonventre, J. V. (1994) The Krüppel associated box-A (KRAB-A) domain of zinc finger proteins mediates transcriptional repression. Proc. Natl. Acad. Sci. USA 91, 4514-4518
12 Alerasool, N., Segal, D., Lee, H., and Taipale, M. (2020) An efficient KRAB domain for CRISPRi applications in human cells. Nature Methods 17, 1093-109
13 Singh, A., Wilson, J. W., Schofield, C. J., and Chen, R. (2020) Hypoxia-inducible factor (HIF) prolyl hydroxylase inhibitors induce autophagy and have a protective effect in an in-vitro ischaemia model. Sci. Rep. 10, 1597-1606
14 Xin, X., Rodrigues, M., Umapathi, M., Kashiwabuchi, F., Ma, T., Babapoor-Farrokhran, S., Wang, S., Hu, J., Bhutto, I., Welsbie, D. S., Duh, E. J., Handa, J. T., Eberhart, C. G., Lutty, G., Semenza, G. L., Montaner, S., and Sodhi, A. (2013) Hypoxic Müller cells promote vascular permeability by HIF-1-dependent up-regulation of angiopoietin-like 4. Proc. Natl. Acad. Sci. USA 110, E3425-E3434
15 Arai, R., Ueda, H., Kitayama, A., Kamiya, N., and Nagamune, T. (2001) Design of the linkers which effectively separate domains of a bifunctional fusion protein. Protein Eng. 14, 529-532
16 Arai, R., Wriggers, W., Nishikawa, Y., Nagamune, T., and Fujisawa, T. (2004) Conformations of variably linked chimeric proteins evaluated by synchroton X-ray small-angle scattering. Proteins 57, 829-838
17 Zhang, J. S., Moncrieffe, M. C., Kaczynski, J., Ellenrieder, V., Prendergast, F. G., and Urrutia, R. (2001) A conserved α-helical motif mediates the interaction of Sp1-like transcriptional repressors with the corepressor mSin3A. Mol. Cell. Biol. 21, 5041-5049
18 Keir, L. S., Firth, R., Aponik, L., Feitelberg, D., Sakimoto, S., Aguilar, E., Welsh, G. I., Richards, A., Usui, Y., Satchell, S. C., Kuzmuk, V., Coward, R. J., Goult, J., Bull, K. R., Sharma, R., Bharti, K., Westenskow, P. D., Michael, I. P., Saleemm M. A., and Friedlander, M. (2017) VEGF regulates local inhibitory complement proteins in the eye and kidney J. Clin. Invest. 127, 199214
19 Gonzalez-Perez, R. R., Xu, Y., Guo, S., Watters, A., Zhou, W., and Leibovich, S. J. (2010) Leptin upregulates VEGF in breast cancer via canonic and non-canonical signalling pathways and NFκβ/HIF-1α activation. Cell. Signal. 22, 1350-1362.
20 Ramanathan, M., Pinhal-Enfield, G., Hao, I., and Leibovich, S. J. (2007) Synergistic up-regulation of vascular endothelial growth factor (VEGF) expression in macrophages by adenosine A2A receptor agonists and endotoxin involves transcriptional regulation via the hypoxia response element of the VEGF promoter. Mol. Biol. Cell 18, 14-23
21 Gao, Z., Herrera-Carrillo, E. and Berkhout, B. (2018) Delineation of the exact transcription termination signal for type 3 polymerase III. Mol. Ther. Nucleic Acids 10, 36-44
22 Kowarz, E., Löscher, D. and Marschalek, R. (2015) Optimized Sleeping Beauty transposons rapidly generate stable transgenic cell lines. Biotechnol. J. 10, 647-653
23 Simpson, D. A. C., Feeney, S., Boyle, C., and Stitt, A. W. (2000) Retinal VEGF mRNA measured by SYBR Green fluorescence: A versatile approach to quantitative PCR. Mol. Vis. 6, 178-183
24 Thakore, P. I., Kwon, J. B., Nelson, C. E., Rouse, D. C., Gemberling, M. P., Oliver, M. L., and Gersbach, C. A. (2018). RNA-guided transcriptional silencing in vivo with S. aureus CRISPR-Cas9 repressors. Nat. Commun. 9, 1674-1682.

It will be understood that various details of the presently disclosed subject matter can be changed without departing from the scope of the subject matter disclosed herein. Furthermore, the foregoing description is for the purpose of illustration only, and not for the purpose of limitation.

1. A nucleic acid, comprising:
a) a nucleotide encoding a CasPhi, wherein there are no amino acid substitutions, or wherein proline residues at amino acids 749 and 753 have been substituted;
b) a nucleotide encoding an amino-terminal linker connected to the amino-terminal end of the CasPhi;
c) a nucleotide encoding a carboxy-terminal linker connected to the carboxy-terminal end of the CasPhi; and
d) a nucleotide encoding a nuclear localization signal (NLS) downstream from the CasPhi and carboxy-terminal linker.

2. The nucleic acid of claim 1, and further comprising a nucleotide encoding a protein domain for facilitating a CRISPR application.

3. The nucleic acid of claim 2, wherein the application is selected from the group consisting of gene editing, imaging, transcriptional activation, and transcriptional repression.

4. The nucleic acid of claim 1, and further comprising a repressor domain or an activator domain.

5. The nucleic acid of claim 1, and further comprising a repressor domain downstream from the CasPhi and carboxy-terminal linker.

6. The nucleic acid of claim 5, wherein the repressor domain is between the carboxy-terminal linker and the NLS.

7. The nucleic acid of claim 5, wherein NLS is between the carboxy-terminal linker and the repressor domain.

8. The nucleic acid of claim 5, wherein the repressor domain is selected from the group consisting of KRAB, SRDX, T1R1, MAD1, and TIEG1.

9. The nucleic acid of claim 8, wherein the repressor domain is a KRAB-domain fusion.

10. (canceled)

13. The nucleic acid of claim 5, and further comprising a second repressor domain.

14. The nucleic acid of claim 13, wherein the second repressor domain is connected to the amino-terminal end of the amino-terminal linker.

15. The nucleic acid of claim 1, and further comprising a first promoter operably connected to the repressor domain or the dCasPhi;

16. The nucleic acid of claim 15, wherein the first promoter is an RNA polymerase II promoter.

17. The nucleic acid of claim 15, and further comprising a guide RNA (gRNA) and a second promoter operably connected to the gRNA.

18. The nucleic acid of claim 1, and further comprising a guide RNA (gRNA) and a second promoter operably connected to the gRNA.

19. The nucleic acid claim 18, wherein the second promoter is an RNA polymerase II promoter.

20-21. (canceled)

22. A composition comprising the nucleic acid of claim 1 and a component for delivery.

23-25. (canceled)

26. A vector comprising the nucleic acid of claim 1.

27. (canceled)
